(19) United States
(12) Patent Application Publication
(10) Pub. No.: US 2008/0033336 A1
(43) Pub. Date: Feb. 7, 2008

(54) Specimen collector with dissolvable coating

(76) Inventor: Jangbir S. Sangha, Overland Park, KS (US)

Correspondence Address: SHUGHART THOMSON & KILROY, PC, 120 WEST 12TH STREET, KANSAS CITY, MO, 64105, US

(21) Appl. No.: 11/704,766

(22) Filed: Feb. 9, 2007

Related U.S. Application Data
(60) Provisional application No. 60/834,874, filed on Aug. 2, 2006.

(51) Int. Cl. A61M 35/00

(52) U.S. Cl. 604/1

(57) ABSTRACT

An improved forensic specimen collector includes a collection matrix coated with a dissolvable coating composition. The coating composition covers the irregular surface of the matrix, filling in the holes and openings to prevent passage of collected material specimens into the interior of the matrix. This facilitates retention of the specimens on the outer surface of the matrix and release of all of the specimens for analysis. The specimens are separated from the surface of the collection matrix by contacting the matrix with a liquid solvent selected to dissolve the coating, thereby releasing the specimen into the solvent. The solvent is selected in accordance with its solubility characteristics to form either a suspension of the specimen in the solvent, or to dissolve the specimen into the solvent to form a solution.

### CROSS-REFERENCE TO RELATED APPLICATION

This application claims priority under 35 U.S.C. 119(e) and 37 C.F.R. 1.78(a)(4) based upon copending U.S. Provisional Application Ser. No. 60/834,874 for COATED DISSOLVABLE SPECIMEN COLLECTION ABSORBENT, filed Aug. 2, 2006 which is expressly incorporated herein by reference.

### BACKGROUND OF THE INVENTION

The present invention is generally concerned with the collection and release of specimens and samples for subsequent analysis. More particularly, it is concerned with forensic specimen collection devices having coatings that promote more efficient collection of specimens of interest and that are dissolvable to permit delivery of all of the captured specimens for analysis without quantitative or qualitative impairment of the analysis.

When a crime has been committed, collection and forensic analysis of evidence are often crucial to determination of guilt or innocence. Trace evidence may be used to associate victims and suspects with each other and with a crime scene. For example, DNA may be recovered from biological samples, such as body fluids, stains, fragments of tissue, bone or hair. Trace evidence may also include nonbiological materials such as residues from gunshots and explosives, unknown chemicals and pharmaceuticals, inks, paints, plastics, fibers, building materials, metals, glass, wood and soil.

Trace evidence is commonly collected using a collector such as a swab having an elongate shaft with a collection tip at one end. The tip is generally covered with an absorbent collection matrix pad consisting of a cellulosic or synthetic resin material such as cotton, rayon or polyester. The material of the collection matrix may be fibrous, or it may be in the form of a net or sponge. While such swabs may appear to present a fairly smooth surface when viewed with the unaided eye, they are actually covered with surface irregularities such as openings, pores and holes which aid in the absorption of liquid specimens and enhance the ability of the swab to retain collected dry specimens.

Depending on the specimen of interest, the swabs may be used in a dry condition to absorb liquids or solids suspended in liquids, or to capture dry particles, or they may be moistened with a suitable liquid to assist in dissolving dried or congealed liquid specimens or in capturing dry specimens. The swabs are swiped, rubbed or dabbed against the specimen of interest to capture or absorb the specimen for collection. Where the specimen is suspended in the air, swabs may be waved through the air to contact and capture the specimens or they may be placed in a selected location to collect particles that settle out of the air. The specimens may also be collected with the assistance of a vacuum device. Once collected, the specimens are retained on or within the collection matrix of the swabs, which are generally placed in individually labeled, airtight specimen containers such as bags or specimen boxes, until they can be transferred to a laboratory for analysis.

In the laboratory, the captured specimens must be separated from the collection matrix for analysis. This may be accomplished by treating the specimen-containing portion of the swab with a solvent to dissolve or elute the specimens into solution with the solvent, which may then be stored in a container such as a flask or test tube awaiting analysis. Portions of the swab not containing specimens may be similarly treated to serve as a control. Where it is not desirable to dissolve the specimens, they may be washed from the swab using a liquid to form a suspension, or they may be physically removed, as by tapping or shaking of the swab or by using mechanical means such as forceps to grasp the specimens one-by-one and remove them or by cutting up the specimen.

While the irregular surface of the swab collection matrix is extremely helpful in capturing and retaining the collected specimens, it may also serve to impair removal of specimens from the swab for the necessary analysis. Where the specimens are particulates which are not to be dissolved, such as skin flakes or hair fragments they may become entrapped within the interior of the collection matrix and become extremely difficult to remove. While some portion of the specimens may be physically removed and available for analysis, the quantity of specimen recovery may be substantially reduced. Accordingly, there is a need to modify the surface of the swabs using a known composition having properties that will not impair the ability of the swabs to collect and retain specimens of interest, and that can be easily removed from the collection matrix along with the specimens, without interfering with subsequent analysis of the specimens.

### SUMMARY OF THE INVENTION

The present invention provides a greatly improved specimen collector having a dissolvable surface coating. The specimen collector includes a collection matrix presenting an outer surface and an interior portion. The outer surface includes irregularities such as holes and openings into the interior portion. When material specimens are collected, some of the smaller specimens pass through the holes and openings into the interior of the matrix, where they become entrapped in the material of the matrix and are difficult to remove. The irregular surface of the collection matrix is modified by application of a dissolvable coating composition.

The coating composition covers the irregular surface of the matrix, filling in the pours or holes and openings. This facilitates retention of the specimens on the outer surface of the matrix and prevents collected specimens from passing through the openings and into the interior portion of the matrix. The specimens may be separated from the surface of the collection matrix by contacting the coating of the matrix with a liquid solvent selected for its ability to dissolve the coating, as well as the specimen, and thereby release the specimen into the solvent or elution buffer. An elution buffer is a predetermined solution of selected compounds designed to provide an optimal environment for the analytes of interest prior to and during analysis. A typical elution buffer is a phosphate buffered solution used for biological samples. The solvent or elution buffer may be selected in accordance with its solubility characteristics to form either a suspension of the specimen in the solvent, or to dissolve the specimen into the solvent to form a solution.

Various objects and advantages of this invention will become apparent from the following description taken in conjunction with the accompanying drawings wherein are set forth, by way of illustration and example, certain embodiments of this invention.

The drawings, including FIGS. 1-10, constitute a part of this specification, include exemplary embodiments of the present invention, and illustrate various objects and features thereof.

### BRIEF DESCRIPTION OF THE DRAWINGS

FIG. 1 is a diagrammatic perspective view of a swab showing a prior art collection tip having no specimen collection matrix surface coating.

FIG. 2 is a diagrammatic perspective view of a test tube containing a prior art swab as shown in FIG. 1 showing specimens captured on the matrix surface.

FIG. 3 is a diagrammatic perspective view showing the collection tip of the prior art swab of FIG. 2 immersed in a solvent liquid, with part of the collected specimens dissolved in the solvent liquid and part of the collected specimens retained by the collection matrix surface.

FIG. 4 is a diagrammatic perspective view of a swab showing a collection tip and specimen collection matrix having a surface coating.

FIG. 5 is a diagrammatic perspective view of a test tube containing a swab as shown in FIG. 4 showing specimens captured on the modified surface.

FIG. 6 is a diagrammatic perspective view showing the collection tip of the swab of FIG. 5 immersed in a solvent liquid, with the surface coating and collected specimens in the process of dissolving in the solvent liquid.

FIG. 7 is a diagrammatic perspective view showing the tube of FIG. 6 with the swab removed and the surface coating and specimen fully dissolved in the solvent.

FIG. 8 is a diagrammatic perspective view of the swab shown in FIG. 4 after the surface coating and specimens have been dissolved off.

FIG. 9 is a greatly enlarged diagrammatic cross-section view taken along line 9-9 of FIG. 8 of a collection matrix showing openings in the outer surface coated by a coating composition.

FIG. 10 is a flow diagram showing the steps in a method of use of a coated specimen collector.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

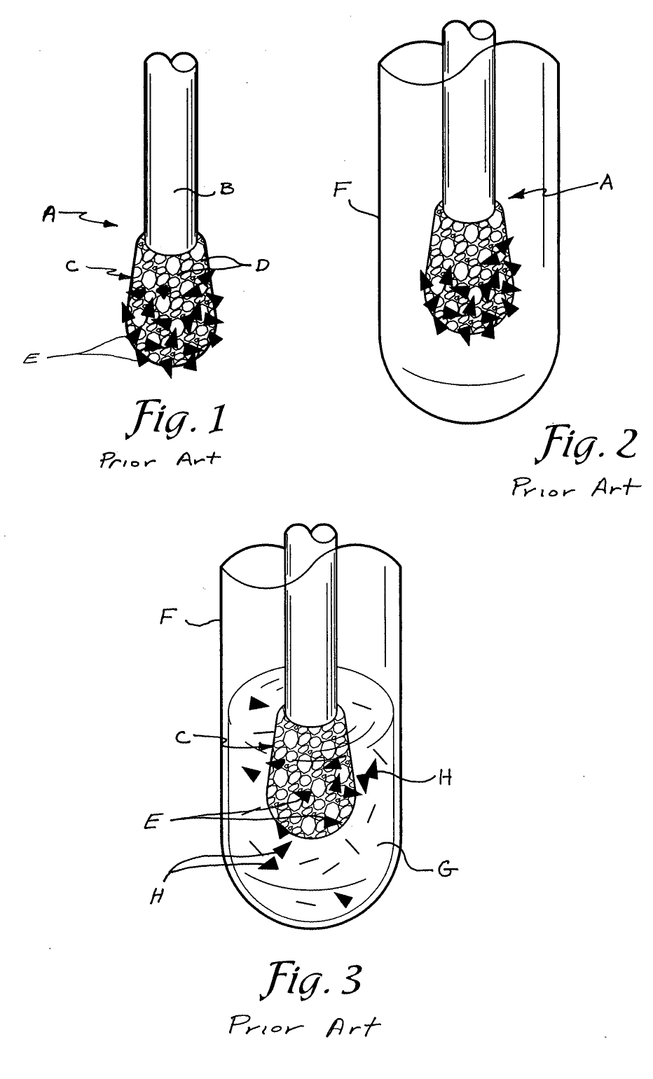

FIG. 1 shows a prior art collection device A comprising a shaft B having a collection matrix C mounted thereon. The collection matrix C is generally rough surfaced in nature and contains various interstitial areas D which can trap a specimen E collected thereon and inhibit elution or removal of the specimen E from the matrix C. Typically, as shown in FIG. 2, specimen collection device A will be inserted into a standard test tube F and an elution buffer or other solution G for dissolving or separating specimen E from collection matrix C is added to tube F. As shown in FIG. 3, such elution of a specimen E is only partially successful with some of specimen E being eluted or dissolved into solution such as shown in FIG. 3 by eluted specimen H which is in solvent or elution buffer G and removed from matrix C. However, a certain amount of specimen E continues to be retained on matrix C as it is bound to collection matrix C by being trapped within interstitial spaces D of matrix C. The present invention, as described hereinafter, overcomes this debility of the prior art by avoiding entrapment of specimen particles within the interstitial spaces of a collection matrix.

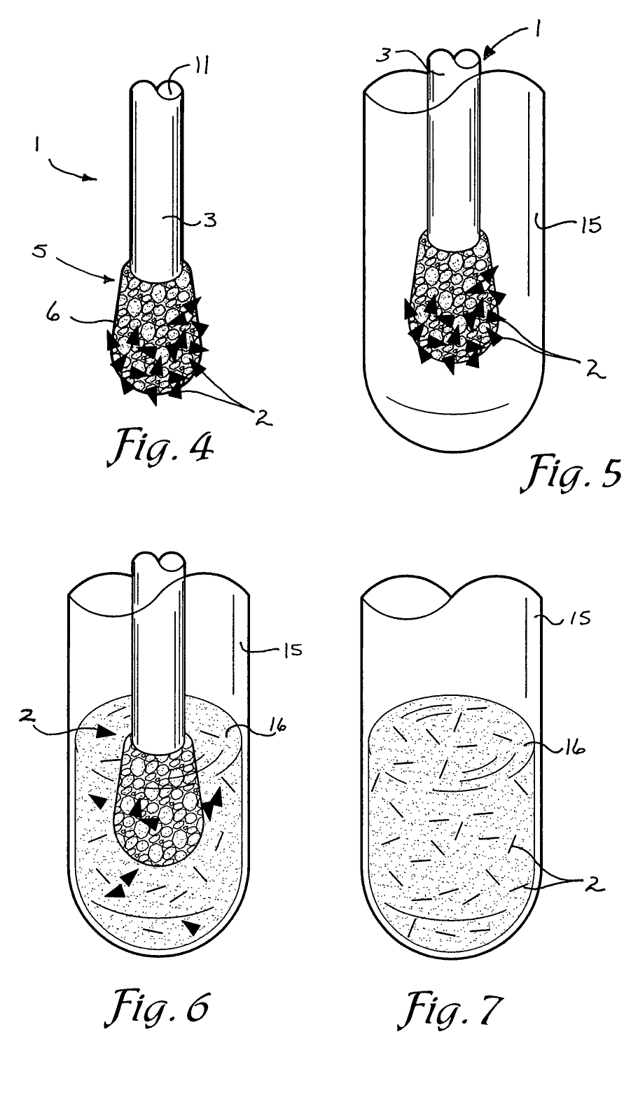

Referring now to drawing FIGS. 4-9, the reference numeral 1 refers to a coated specimen collector 1, which is depicted in FIGS. 4-6 in association with a quantity of material samples or specimens 2. The collector 1 is depicted in the form of a swab, having an elongate shaft 3, with a pair of ends. The shaft 3 may be constructed of wood, paper, resin, glass or metal, and it may be flat, tubular, cylindrical, tab-shaped or it may be of any other suitable material and configuration.

One end of the shaft 3 is connected to a head or tip portion 5. As shown in FIGS. 4 and 5, the tip 5 is covered with a collection matrix 6. The collection matrix 6 is generally formed from an absorbent cellulosic or synthetic resinous material such as, for example, cotton, rayon or polyester. The matrix 6 may also be selected from any other suitable material that will not interfere with collection of a specimen of interest and retention of the collected specimen on the collector head 5, that is compatible with the specimen of interest and the coating, and that will not interfere with release of the specimen or dissolution of the matrix coating. The material may be in the form of a fibrous wadding that is wrapped around the end of the shaft 3, or it may be in the form of a small pad, sponge or net that is attached to the shaft 3. An inert adhesive composition may also be employed to retain the material in place on the shaft 3. It is foreseen that the collector 1 may be constructed without a shaft 3, so that it consists of only the collection matrix 6 in the form of a ball, pad, wipe, stick or net.

Regardless of the overall configuration of the collector 1, the collection matrix 6 (FIG. 9) presents an irregular outer surface 11 having a plurality of holes, pores or openings 12 in communication with an interior portion 13 of the matrix. The matrix 6 is generally covered with a quantity of a dissolvable coating composition 14, (FIG. 9) which blocks or fills some or all of the openings 12, thereby preventing passage of at least some of the specimens 2 into the matrix interior 13.

The coating composition 14 is selected for solubility characteristics that enable it to dissolve in a preselected extraction or elution solvent. As used herein, the term “dissolve” means the chemical process of solvation (including hydration), in which a solute is dissolved in a solvent, creating a solution. The solute coating composition 14 herein is dissolved in an extraction or elution solvent to create a solution containing solubilized coating composition and solvent. The coating composition 14 and the solvent may be selected for solvation under ambient conditions. It is also foreseen that the coating composition and/or the solvent may be preselected for solvation under conditions that require the temperature and/or pressure to be manipulated, i.e., by raising and/or lowering in order to control the induction or rate of solvation.

The coating composition 14 is also selected based on consideration of additional factors such as the composition of the collection matrix, the surface irregularities thereon, the specimen of interest 2 to be collected, the conditions under which the specimen is to be collected, and the potential interactions between such factors. Some exemplary coating compositions are proteins such as albumin, salt solutions, carboxymethyulcellulose, polysaccharides, glucose, phosphate buffer solution, gelatin and mixtures thereof. Where the coated specimen collector 1 may be used to collect cells, for example from the mouth of a live subject, the coating composition is selected from materials generally regarded as safe for human contact or ingestion.

The concentration of the coating composition 14 is also preselected based on the foregoing considerations and the ability of the composition 14 to modify the outer surface 11 of the collection matrix 6 to promote more efficient collection of the analytes of interest, such as, for example, DNA or other trace evidence, and of the surface coating 14 to dissolve into solution with the solvent and bring along all of the captured specimens 2 into an elution vessel or container such as a test tube 15 (FIGS. 5-7).

A known quantity of the coating composition 14 may be applied to the irregular surface 11 of the collection matrix 6 by passive absorption in the form of a liquid, or deposition of a vapor or gas which is allowed to dry prior to use. The coating 14 may also be applied by electrochemical assisted deposition or any other suitable method. In certain embodiments, the coating 14 may be formulated to retain a tacky surface after drying to facilitate collection of specimens of interest 2. It is also foreseen that the coating composition 14 may be applied to specimen collector heads 5 that are not porous or irregular, in order to facilitate release of captured specimens 2. The quantity of coating composition 14 applied to the collection matrix 6 is known and may be varied in accordance with the concentration of the coating 14, the composition of the collection matrix 6 and the solvent, the specimen 2 to be collected, or any combination of the foregoing.

The solvent 16, shown in FIG. 6 dissolving the coating 14 to form a solution, is also selected for solubility characteristics that permit solvation of the coating composition 14 so that it will elute the coating from the collection matrix 6. It may also be preselected to dissolve the specimens 2 as well. The solvent may be in the form of a liquid, or it may be in the form of a vapor or a gas. The solvent may be organic, such as an aqueous solvent, or it may be organic and it may be in the form of a solution or mixture of suitable solvent compositions.

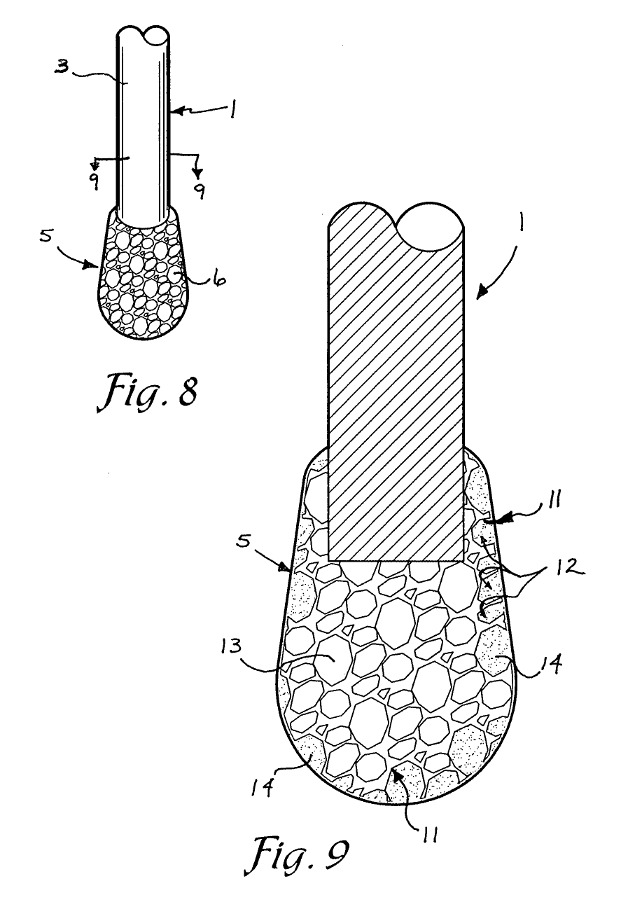

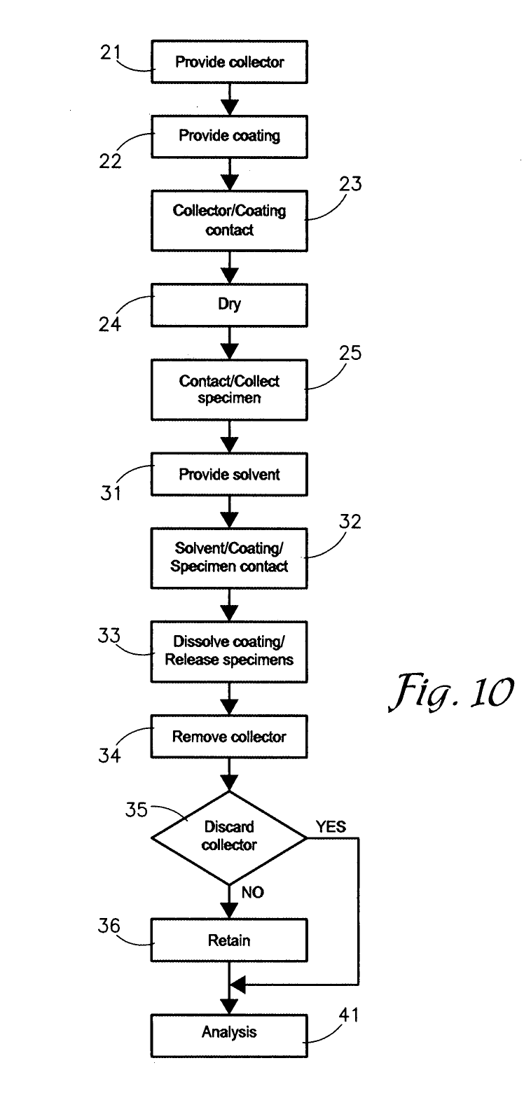

In the steps of a method of use (FIG. 10), a first step 21 is providing a specimen collector 1. In a next step 22, a known quantity of a coating composition 14 is provided 22. In a next step 23, the head 5 of the specimen collector 1 is contacted with a quantity of a coating composition 14 in the form of a liquid, vapor or gas to cause the coating 14 to deposit itself on the outer surface 11 of the collection matrix 6 in covering relation to at least some of the irregularities or openings 12 in the matrix surface 11 (FIGS. 8 and 9). As an example, if the specimen collector 1 comprises a cotton swab, the swab head 5 including the cotton collection matrix 6 may be coated with a gelatin solution, a solution of carboxymethylcellulose, a glucose solution, a phosphate buffer solution or a polysaccharide solution. In the next step 24, the coating composition 14 is permitted to dry on the matrix outer surface 11 for a period of time. It is foreseen that the collector 1 may also be used in a wet or tacky condition. If the specimen collector 1 comprises a rayon swab, the swab head 5, including the rayon collection matrix 6 may be coated with a gelatin solution, albumin solution or a carboxymethylcellulose solution compatible with the solvent 16.

In a next step 25, the coated collector matrix 6 is next used to contact a specimen of interest 2, which is collected and retained on the coated surface of the collector 1 (FIG. 4). A quantity of a preselected solvent is next provided 31. In the next step 32, the coated collection matrix outer surface 11 and collected specimens 2 are contacted with a quantity of the selected solvent 16. This may be accomplished by introducing the collector 1 with collected specimens 2 into a container or vessel 15 such as a test tube (FIG. 5). A quantity of a solvent 16 may be present in the tube 15 when the collector 1 is introduced, or the solvent 16 may be added to the tube 15 subsequently. In the next step 33, the solvent 16 is permitted to dissolve the coating 14 and thereby release the specimens 2 from the outer surface 11 of the collection matrix 6 and into the solvent 16 (FIG. 6). The collector 1 is next removed 34 from the vessel 15 (FIG. 7), leaving a solution formed by the coating composition 14 and the solvent 16. The specimens 2 may be dissolved in the solvent solution as shown in FIG. 4, or they may be suspended in the solution in an undissolved state. The specimen collector 1 with its collection matrix 6 with coating composition 14 and specimens 2 now fully eluted (FIG. 7) may be discarded 34 or retained 35. In the next step 41, an analytical procedure is performed on the released specimens 2. This step may be repeated using different reagents and procedures until the specimens 2 have been identified and characterized to the fullest possible extent.

It is to be understood that while certain forms of the present invention have been illustrated and described herein, it is not to be limited to the specific forms or arrangement of parts described and shown.

The following is claimed and desired to be secured by Letters Patent:

1. A specimen collector, comprising:
a. a specimen collection matrix having an outer surface and an interior portion;
b. said outer surface including a plurality of openings into said collection matrix interior portion;
c. a coating on said collection matrix outer surface for covering at least some of said openings to retain a collected specimen on said outer surface and prevent said specimen from passing through said openings and into said matrix interior portion; and
d. said coating being soluble when contacted by a solvent to thereby release said specimen into said solvent.

2. The specimen collector as set forth in claim 1, wherein:
a. said specimen collection matrix is constructed of a cellulosic material.

3. The specimen collector as set forth in claim 1, wherein:
a. said specimen collection matrix is constructed of cotton.

4. The specimen collector as set forth in claim 1, wherein:
a. said specimen collection matrix is constructed of rayon.

5. The specimen collector as set forth in claim 1, wherein:
a. said specimen collection matrix is constructed of a synthetic resin material.

6. The specimen collector as set forth in claim 1, wherein:
a. said specimen collection matrix is constructed of polyester.

7. The specimen collector as set forth in claim 1, wherein:
a. said coating is a composition selected from the group consisting essentially of: gelatin, carboxymethylcellulose, albumin, a salt; a polysaccharide, and glucose.

8. The specimen collector as set forth in claim 1, wherein:
a. said coating is soluble in an aqueous solvent.

9. The specimen collector as set forth in claim 1, wherein:
b. said coating is soluble in an organic solvent.

10. The specimen collector as set forth in claim 1, wherein:
a. said solvent is selected to dissolve said collected specimen into said solvent and form a solution.

11. The specimen collector as set forth in claim 1, wherein:
a. said collected specimen is insoluble in said solvent.

12. The specimen collector as set forth in claim 1, wherein:
a. said solvent is an aqueous liquid or vapor.

13. The specimen collector as set forth in claim 1, wherein:
a. said solvent is an organic liquid or vapor.

14. A specimen collection swab, comprising:
a. an elongate shaft having a collection tip with a collection matrix;
b. said specimen collection matrix having an outer surface and an interior portion;
c. said outer surface including a plurality of openings into said collection matrix interior portion;
d. a coating composition coating said collection matrix outer surface for covering at least some of said openings to retain a collected specimen on said outer surface and prevent said specimen from passing through said openings and into said matrix interior portion; and
d. said coating composition being soluble when contacted by a solvent to thereby dissolve said coating from said collection matrix outer surface and release said specimens into said solvent.

15. The specimen collection swab as set forth in claim 14, wherein:
a. said collection matrix is constructed of cotton; and
b. said coating composition is selected from the group consisting essentially of: gelatin and carboxymethylcellulose.

16. The specimen collection swab as set forth in claim 14, wherein:
a. said collection matrix is constructed of rayon; and
b. said coating composition is selected from the group consisting essentially of: gelatin, albumin and carboxymethylcellulose.

17. The specimen collection swab as set forth in claim 14, wherein:
a. said collection matrix is constructed of polyester; and
b. said coating composition is selected from the group consisting essentially of: gelatin and carboxymethylcellulose.

18. The specimen collection swab as set forth in claim 14, wherein:
a. said collection matrix is constructed of cellulose; and
b. said coating composition is selected from the group consisting essentially of a polysaccharide composition and a glucose solution.

19. A collection tip for use with a specimen collection device, comprising:
a. an absorption matrix having an outer surface including a plurality of openings and an interior portion;
b. a coating on said absorption matrix for covering at least some of said openings to retain collected specimens on said outer surface and prevent specimens from passing through said openings and into said matrix interior portion; and
c. said coating composition being soluble in a liquid solvent to thereby dissolve said coating from said collection matrix outer surface and release said specimens into said liquid solvent.

20. A method of collecting and releasing a specimen from a specimen collection device, comprising;
a. providing a specimen collector including a specimen collection matrix having an outer surface including a plurality of openings and an interior surface;
b. providing a quantity of a coating composition;
c. contacting said matrix outer surface with said coating composition for covering at least some of said openings to retain collected specimens on said outer surface and prevent specimens from passing through said openings and into said matrix interior portion;
d. enabling said coating composition to dry on said matrix outer surface;
e. using said specimen collector to contact a specimen of interest to thereby collect said specimen on said coated collection matrix surface;
f. providing a quantity of a solvent solution in a vessel;
g. contacting said coated collection matrix outer surface and said collected specimens with a quantity of said solvent solution; and
h. enabling said coating to dissolve from said matrix outer surface and release said specimens from said outer surface and into said solvent.

21. The method as set forth in claim 20, wherein said coating composition is provided in the form of a liquid.

22. The method as set forth in claim 21, further including the step of:
a. immersing said matrix outer surface in said liquid solvent solution.

23. The method as set forth in claim 20, wherein said coating composition is provided in the form of a vapor.

24. The method as set forth in claim 20, further including the steps of:
a. retaining said solvent solution including said dissolved coating and released specimens in said vessel.

25. The method as set forth in claim 20, further including the step of:
a. performing an analytical procedure on said released specimens.
